(19) United States
(12) Patent Application Publication
(10) Pub. No.: US 2005/0085489 A1
(43) Pub. Date: Apr. 21, 2005

(54) Pharmaceutical composition comprising a glitazone and a 4-oxobutanoic acid, and the use thereof for treating diabetes

(76) Inventor: Gerard Moinet, Orsay (FR)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/501,069

(22) PCT Filed: Dec. 16, 2002
(86) PCT No.: PCT/EP02/14311

(30) Foreign Application Priority Data
Jan. 11, 2002 (FR) 0200335

(51) Int. Cl. A61K 31/445; A61K 31/44; A61K 31/401; A61K 31/381; A61K 31/192

(52) U.S. Cl. 514/256; 514/355; 514/423; 514/317; 514/448; 514/461; 514/569; 514/570

(57) ABSTRACT

The present invention relates to a pharmaceutical composition comprising, as active principles, a 4-oxobutanoic acid and a glitazone, in combination with one or more pharmaceutically acceptable excipients. These compositions are particularly suitable for treating diabetes.

The present invention relates to a pharmaceutical composition comprising, as active principles, a 4-oxobutanoic acid described in WO 98/07681 and a glitazone.

The invention also relates to the use of a 4-oxobutanoic acid and a glitazone for the preparation of a medicinal preparation for reducing hyperglycaemia, more particularly the hyperglycaemia of non-insulin-dependent diabetes.

Diabetes is a chronic disease that has various pathological manifestations. It is accompanied by disorders of lipid and sugar metabolism and circulatory disorders. In many cases, diabetes tends to progress to a variety of pathological complications. Thus, it is necessary to find the treatment that is suited to each individual suffering from diabetes.

Insulin resistance syndrome (syndrome X) is characterised by a reduction in the action of insulin (Presse Medicale, 26, No. 14, (1997), 671-677) and is involved in a great many pathological conditions, such as diabetes and more particularly non-insulin-dependent diabetes, dyslipidaemia, obesity, arterial hypertension and also certain microvascular and macrovascular complications, for instance atherosclerosis, retinopathies, nephropathies and neuropathies.

4-Oxobutanoic acids have already been described in patent application WO 98/07681 for treating diabetes. Some of these compounds act on the short-lived early secretion of insulin.

The combination of a glitazone, such as troglitazone, and a biguanide antidiabetic agent, more particularly metformin, has already been described for the treatment of diabetes (U.S. Pat. No. 6,011,049 from the Warner Lambert company).

The combination of a glitazone with an insulinosecretagogue in the treatment of diabetes has likewise been described. This is the combination of thiazolidinedione and sulfonylurea (WO 98/57649 and WO 99/03476).

The specific combination of a glitazone with a 4-oxobutanoic acid has not been described and offers particular advantages, especially the absence of weight gain and/or of haemodilution.

Thus, one object of the present invention is to propose a composition for significantly improving the use of glucose.

A further object of the invention is to propose a composition that is suitable for treating diabetes by displaying considerable action on the metabolic syndrome of insulin resistance.

A final object of the invention is to propose a composition that is particularly suitable for diabetics at the various stages of the disease.

These objects and others are achieved by the present invention, which relates to a pharmaceutical composition comprising, as active principles, at least one glitazone and at least one compound of the formula (I), in combination with one or more pharmaceutically acceptable excipients.

This composition is particularly suitable for treating diabetes, more particularly non-insulin-dependent diabetes. It is particularly suitable for reducing the hyperglycaemia of non-insulin-dependent diabetes.

It is also particularly suitable for treating pathologies associated with insulin resistance syndrome, such as, especially, dyslipidaemia, obesity, arterial hypertension, and microvascular and macrovascular complications, for instance atherosclerosis, retinopathies, nephropathies and neuropathies.

The compound of the formula (I) is defined as follows: 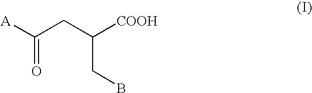
in which the groups A and B are chosen, independently of each other, from:
a mono-, bi- or tricyclic aryl group containing from 6 to 14 carbon atoms;
a heteroaromatic group chosen from pyridyl, pyrimidyl, pyrrolyl, furyl and thienyl groups;
an alkyl group containing from 1 to 14 carbon atoms;
a cycloalkyl group containing from 5 to 8 carbon atoms;
a saturated heterocyclic group chosen from tetrahydrofuryl, tetrahydropyranyl, piperidyl and pyrrolidinyl groups;
the groups A and B possibly bearing 1 to 3 substituents chosen from a C₁-C₆ alkyl group, a C₁-C₆ alkoxy group, a C₆-C₁₄ aryl group, a heteroaryl group chosen from pyridyl, pyrimidyl, pyrrolyl, furyl and thienyl, a (C₆-C₁₄)aryl(C₁-C₆)alkyl group, a (C₆-C₁₄)aryl(C₁-C₆)alkyl(C₆-C₁₄)aryl group, a halogen or a trifluoromethyl, trifluoromethoxy, cyano, hydroxyl, nitro, amino, carboxyl, (C₁-C₆)alkoxycarbonyl, carbamoyl, (C₁-C₆)alkylsulfonyl, sulfoamino, (C₁-C₆)alkylsulfonylamino, sulfamoyl or (C₁-C₆)alkylcarbonylamino group;
or two of the substituents forming a methylenedioxy group, a solvate thereof or a salt of this acid.

In a preferred embodiment of the invention, the 4-oxobutanoic acids are those of the formula (I) in which A and B are chosen from aryl groups.

Examples of aryl groups that may be mentioned include phenyl, α-naphthyl, β-naphthyl and fluorenyl groups.

The C₁-C₆ alkyl groups may be linear or branched. Examples that may be mentioned include methyl, ethyl, propyl, isopropyl, butyl, isobutyl, tert-butyl and pentyl groups.

The C₁-C₆ alkoxy groups may also be linear or branched.

Examples that may be mentioned include methoxy, ethoxy, propoxy, isopropoxy, butoxy and isobutoxy groups.

The halogens may be chosen from fluorine, chlorine, bromine and iodine.

The present invention also includes the tautomeric forms of the compounds of the general formula (I), the enantiomers, diastereoisomers and epimers of these compounds, and also the solvates thereof.

Examples of salts of the compounds of the general formula (I) include pharmacologically acceptable salts, such as the sodium salts, potassium salts, magnesium salts, calcium salts, amine salts and other salts of the same type (aluminium, iron, bismuth, etc.).

In a preferred embodiment, the 4-oxobutanoic acids are chosen from:
2-benzyl-4-(4-methoxyphenyl)₄-oxobutanoic acid
2-benzyl-4-(4-fluorophenyl)-4-oxobutanoic acid
2-cyclohexylmethyl-4-(4-methoxyphenyl)-4-oxobutanoic acid
2-benzyl-4-phenyl-4-oxobutanoic acid
2-(β-naphthylmethyl)-4-phenyl-4-oxobutanoic acid
2-benzyl-4-(β-naphthyl)-4-oxobutanoic acid
2-[(4-chlorophenyl)methyl]-4-(4-methoxyphenyl)-4-oxobutanoic acid
2-benzyl-4-(4-methylphenyl)-4-oxobutanoic acid
4-(4-fluorophenyl)-2-[(4-methoxyphenyl)methyl]-4-oxobutanoic acid
2-benzyl-4-(3,4-methylenedioxyphenyl)-4-oxobutanoic acid
2-benzyl-4-cyclohexyl-4-oxobutanoic acid
4-phenyl-2-[(tetrahydrofur-2-yl)methyl]4-oxobutanoic acid, the solvates, enantiomers and salts of these acids.

The 4-oxobutanoic acid is advantageously chosen from:
(−)-2-benzyl-4-(4-methoxyphenyl)-4-oxobutanoic acid
(+)-2-benzyl-4-(4-methoxyphenyl)-4-oxobutanoic acid
(−)-2-benzyl-4-(4-fluorophenyl)₄-oxobutanoic acid
(+)-2-benzyl-4-(4-fluorophenyl)₄-oxobutanoic acid.

The glitazones are a family of antidiabetic agents which are characterised as being aralkylthiazolidine-2,4-dione derivatives or analogues thereof. The glitazones are preferably compounds of the general formula (II) below: 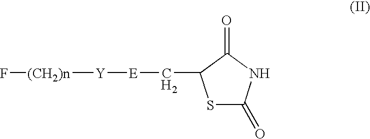
in which:
E represents a monocyclic, bicyclic or tricyclic aromatic hydrocarbon-based structure that can include one or more hetero atoms, this structure possibly being substituted by at least one (C₁-C₆) alkyl or acetyl radical, or possibly forming a 5- or 6-membered ring with the methylene radical attached to Y,
n is equal to 1, 2 or 3,
Y represents an oxygen atom, an —NHCO—, —CONH— or —CO— function; and
F features an amino group or an aromatic or non-aromatic, cyclic or bicyclic hydrocarbon-based group, optionally containing a hetero atom chosen from oxygen and nitrogen, the amino and hydrocarbon-based groups possibly containing at least one substitution chosen from a (C₁-C₆) alkyl radical, a halogen atom, an aryl or heteroaryl radical, an acetyl radical and a trifluoromethyl radical,
and the pharmaceutically acceptable salts thereof.

In the text hereinabove, among the aromatic radicals E that may be mentioned as homocarbon-based structures are the phenyl, α-naphthyl, β-naphthyl, anthracenyl and fluorenyl radicals. Among the heterocyclic aromatic radicals that may be mentioned are pyridyl and the quinolinyl and phenoxazole rings.

In the text hereinabove, among the aromatic radicals F that may be mentioned as homocarbon-based structures are the phenyl, α-naphthyl, β-naphthyl, anthracenyl and fluorenyl radicals. Among the heterocyclic aromatic radicals that may be mentioned are pyridyl and the quinolinyl, benzimidazole, oxazole and phenoxazole rings.

The preferred glitazones have the following formulae:

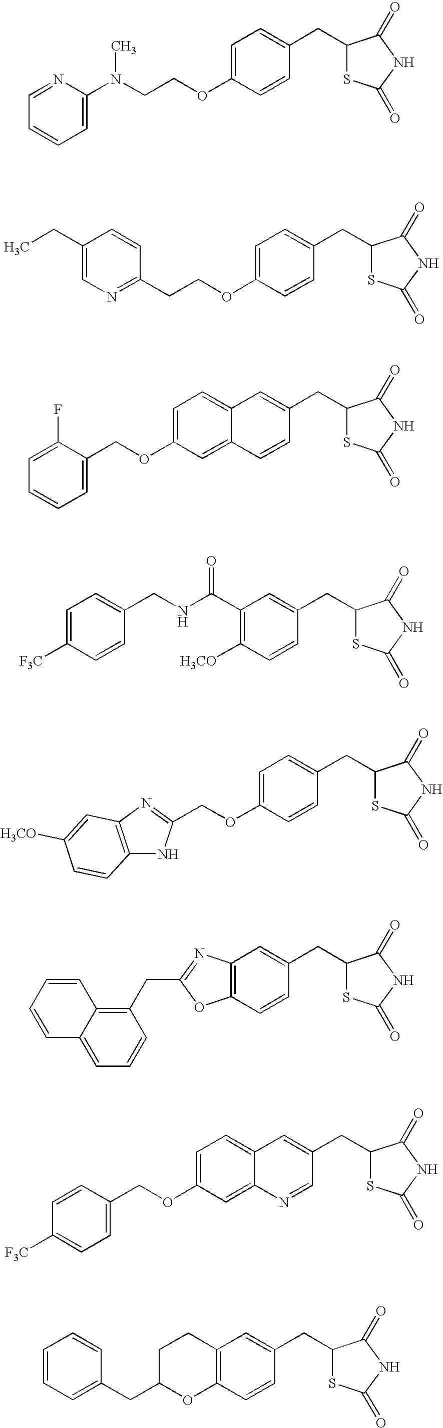

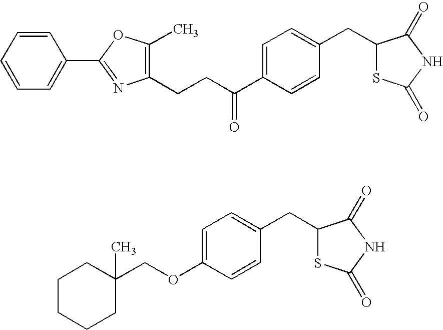

These compounds have the following respective trade names or codes: rosiglitazone (or Avandia®) from the GlaxoSmithkline company, pioglitazone (or Actos®) from the Takeda company, isaglitazone (or MCC 555) from the Mitsubishi company, KRP 297 from the Kyorin company, CS 011 from the Sankyo company, T 174 from the Tanabe company, NP 0110 from the Nippon Chemiphar company, englitazone from the Pfizer company, darglitazone from the Pfizer company and ciglitazone from the Takeda company.

The glitazone is advantageously chosen from rosiglitazone, pioglitazone, isaglitazone (MCC555) and KRP 297.

The compositions of the invention comprise therapeutically effective amounts of the various active principles. The ratios of the respective amounts of glitazone and the compound of the formula (I) thus vary in consequence. Specifically, the dose of each active principle will vary as a function of the severity of the disease, the frequency of administration, the choice of combined active principles and other factors systematically considered by the prescribing doctor for the patient suffering from diabetes.

To give an order of magnitude, the weight ratio of glitazone to the compound of the formula (I) ranges from 10⁻³ to 40, preferably from 10⁻³ to 10 and better still from 10⁻³ to 1.

The compositions of the invention are preferably administered parenterally, or better still orally, although the other routes of administration, for instance such as rectal administration, are not excluded.

If oral administration is envisaged, the compositions of the invention are in the form of gel capsules, effervescent tablets, coated or uncoated tablets, sachets, sugar-coated tablets, drinkable vials or solutions, microgranules or sustained-release forms.

If parenteral administration is envisaged, the compositions of the invention are in the form of injectable solutions and suspensions packaged in vials or bottles for slow venous infusion.

The forms for oral administration are prepared by mixing the active substance with various types of excipients or vehicles, such as fillers, disintegration (or crumbling) agents, binders, dyes, flavour enhancers and the like, followed by shaping the mixture.

The dye can be any dye authorised for pharmaceutical use.

Examples of flavour enhancers include cocoa powder, mint, borneol and cinnamon powder.

Examples of binders that may be mentioned are polyvinylpyrrolidone, hydroxypropylmethylcellulose, alginic acid, carbomer, carboxymethylcellulose, dextrin, ethylcellulose, starch, sodium alginate, polymethacrylate, maltodextrin, liquid glucose, magnesium aluminium silicate, hydroxyethylcellulose, hydroxypropylcellulose, ethylcellulose, methylcellulose and guar gum.

It is possible to use alginic acid, sodium carboxymethylcellulose, colloidal silicon dioxide, croscarmellose sodium, crospovidone, guar gum, magnesium aluminium silicate, methylcellulose, microcrystalline cellulose, cellulose powder, pre-gelatinised starch, sodium alginate or sodium starch glycolate as disintegration agent.

The fillers are, for example, cellulose, lactose, calcium hydrogen phosphate and microcrystalline cellulose.

The tablets can be obtained in a conventional manner by compressing granules in the presence of one or more lubricants. Suitable lubricants are calcium stearate, glyceryl monostearate, glyceryl palmitostearate, hydrogenated castor oil, hydrogenated plant oil, light mineral oil, magnesium stearate, polyethylene glycol, sodium benzoate, sodium lauryl sulfate, stearyl sodium fumarate, stearic acid, talc and zinc stearate. These tablets can then be coated using polymers in solution or suspension, such as hydroxypropyl-methylcellulose or ethylcellulose.

The granules used to do this are prepared, for example, by using the wet granulation process starting with a mixture of the active principles with one or more excipients such as a binder, a crumbling agent (or disintegration agent) and a filler.

To obtain hard capsules, the mixture of the active principles with a suitable filler (for example lactose) is incorporated into empty gelatine capsules optionally in the presence of a lubricant such as magnesium stearate, stearic acid, talc or zinc stearate.

Gel capsules or soft capsules are prepared by dissolving the active principles in a suitable solvent (for example polyethylene glycol), followed by incorporation into soft capsules.

The forms for parenteral administration are obtained in a conventional manner by mixing the active principles with buffers, stabilisers, preserving agents, solubilising agents, isotonicity agents and suspension agents. In accordance with the known techniques, these mixtures are subsequently sterilised and then packaged in the form of intravenous injections.

As buffer, a person skilled in the art can use buffers based on organo-phosphate salts.

Examples of suspension agents include methylcellulose, hydroxyethylcellulose, hydroxypropylcellulose, acacia and sodium carboxymethylcellulose.

Examples of solubilising agents include castor oil solidified with polyoxyethylene, polysorbate 80, nicotinamide or macrogol.

In addition, stabilisers that are useful according to the invention are sodium sulfite and sodium metasulfite, while mention may be made of sodium p-hydroxybenzoate, sorbic acid, cresol and chlorocresol as preserving agents. For the preparation of an oral solution or suspension, the active principles are dissolved or suspended in a suitable vehicle with a dispersant, a wetting agent, a suspension agent (for example polyvinylpyrrolidone), a preserving agent (such as methylparaben or propylparaben), a flavour enhancer or a dye.

For the preparation of suppositories, the active principles are mixed in a manner that is known per se with a suitable base constituent, such as polyethylene glycol or semisynthetic glycerides.

For the preparation of microcapsules, the active principles are combined with suitable diluents, suitable stabilisers, agents that promote the sustained release of the active substances or any other type of additive for the formation of a central core which is then coated with a suitable polymer (for example a water-soluble resin or a water-insoluble resin). The techniques known to those skilled in the art will be used for this purpose.

The microcapsules thus obtained are then optionally formulated in suitable dosage units.

The present invention also relates to the use of a glitazone in combination with a compound of the formula (I) as defined above for the preparation of a medicinal combination for treating diabetes, more particularly non-insulin-dependent diabetes.

According to another of its aspects, the invention relates to the use of a glitazone in combination with the said compound of the formula (I) for the preparation of a medicinal combination for reducing the hyperglycaemia of non-insulin-dependent diabetes.

The present invention also relates to a process for treating diabetes, more particularly non-insulin-dependent diabetes, in a mammal, comprising the administration to the said mammal of the composition according to the present invention.

The glitazones are generally administered in doses ranging from about 1 mg to about 2500 mg per day and more specifically from about 2 mg to about 1000 mg per day. The preferred glitazone is rosiglitazone, and is used in doses ranging from about 1 mg to about 10 mg per day. Another preferred glitazone is pioglitazone, and is administered in doses ranging from about 50 mg to about 200 mg per day.

As regards the compound of the formula (I), it is generally administered in doses ranging from about 25 to 400 mg per day.

If the glitazone and the compound of the formula (I) are incorporated into the same unit dose, the unit dose preferably comprises from 1 mg to 1 g of glitazone and from 12.5 to 400 mg of a compound of the formula (I) (the dose depends especially on the active agents under consideration).

Naturally, the dosage depends on the active agent under consideration, the mode of administration, the therapeutic indication and the age and condition of the patient.

Concrete but non-limiting examples of the invention will now be presented. The percentages given are expressed on a weight basis, except where otherwise mentioned.

### EXAMPLE 1

A tablet having the following composition is prepared:

| | Mass in mg | % by weight |
|---|---|---|
| Compound P* | 50 | 46.3 |
| Rosiglitazone | 2 | 1.9 |
| Microcrystalline cellulose | 15 | 13.9 |
| Fine lactose powder | 21 | 19.4 |
| Hydroxypropylcellulose | 7 | 6.5 |
| Croscarmellose sodium | 10 | 9.3 |
| Colloidal silica (Aérosil ®) | 1.5 | 1.4 |
| Mg stearate | 1.5 | 1.4 |

*Compound P: (+)-2-benzyl-4-(4-fluorophenyl)-4-oxobutanoic acid.

### EXAMPLE 2

A tablet having the following composition is prepared:

| | Mass in mg | % by weight |
|---|---|---|
| Compound P* | 50 | 39.4 |
| Rosiglitazone | 4 | 3.1 |
| Microcrystalline cellulose | 17 | 13.4 |
| Fine lactose powder | 26 | 20.5 |
| Hydroxypropylcellulose | 11 | 8.7 |
| Croscarmellose sodium | 15 | 11.8 |
| Colloidal silica (Aérosil ®) | 2 | 1.6 |
| Mg stearate | 2 | 1.6 |

*Compound P: (+)-2-benzyl-4-(4-fluorophenyl)-4-oxobutanoic acid.

### EXAMPLE 3

A tablet having the following composition is prepared:

| | Mass in mg | % by weight |
|---|---|---|
| Compound P* | 100 | 56.5 |
| Rosiglitazone | 2 | 1.1 |
| Microcrystalline cellulose | 22 | 12.4 |
| Fine lactose powder | 24 | 13.6 |
| Hydroxypropylcellulose | 12 | 6.8 |
| Croscarmellose sodium | 13 | 7.3 |
| Colloidal silica (Aérosil ®) | 2 | 1.1 |
| Mg stearate | 2 | 1.1 |

*Compound P: (+)-2-benzyl-4-(4-fluorophenyl)-4-oxobutanoic acid.

### EXAMPLE 4

A tablet having the following composition is prepared:

| | Mass in mg | % by weight |
|---|---|---|
| Compound P* | 100 | 49.8 |
| Rosiglitazone | 4 | 2 |
| Microcrystalline cellulose | 24 | 11.9 |
| Fine lactose powder | 33 | 16.4 |
| Hydroxypropylcellulose | 15 | 7.5 |
| Croscarmellose sodium | 19 | 9.5 |
| Colloidal silica (Aérosil ®) | 3 | 1.5 |
| Mg stearate | 3 | 1.5 |

*Compound P: (+)-2-benzyl-4-(4-fluorophenyl)-4-oxobutanoic acid.

### EXAMPLE 5

A tablet having the following composition is prepared:

| | Mass in mg | % by weight |
|---|---|---|
| Compound P* | 200 | 62.7 |
| Rosiglitazone | 2 | 0.6 |
| Microcrystalline cellulose | 32 | 10.0 |
| Fine lactose powder | 40 | 12.5 |
| Hydroxypropylcellulose | 15 | 4.7 |
| Croscarmellose sodium | 22 | 6.9 |
| Colloidal silica (Aérosil ®) | 4 | 1.3 |
| Mg stearate | 4 | 1.3 |

*Compound P: (+)-2-benzyl-4-(4-fluorophenyl)-4-oxobutanoic acid.

### EXAMPLE 6

A tablet having the following composition is prepared:

| | Mass in mg | % by weight |
|---|---|---|
| Compound *P | 200 | 58.3 |
| Rosiglitazone | 4 | 1.2 |
| Microcrystalline cellulose | 35 | 10.2 |
| Fine lactose powder | 49 | 14.3 |
| Hydroxypropylcellulose | 20 | 5.8 |
| Croscarmellose sodium | 27 | 7.9 |
| Colloidal silica (Aérosil ®) | 4 | 1.2 |
| Mg stearate | 4 | 1.2 |

*Compound P: (+)-2-benzyl-4-(4-fluorophenyl)-4-oxobutanoic acid.

1. Pharmaceutical composition comprising, as active principles, (i) at least one glitazone and (ii) at least one compound of the formula (I), in combination with one or more pharmaceutically acceptable excipients, the compound of the formula (I) being defined as follows: 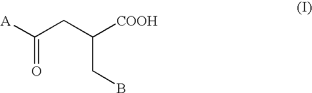
in which the groups A and B are chosen, independently of each other, from:
a mono-, bi- or tricyclic aryl group containing from 6 to 14 carbon atoms;
a heteroaromatic group chosen from pyridyl, pyrimidyl, pyrrolyl, furyl and thienyl groups;
an alkyl group containing from 1 to 14 carbon atoms;
a cycloalkyl group containing from 5 to 8 carbon atoms;
a saturated heterocyclic group chosen from tetrahydrofuryl, tetrahydropyranyl, piperidyl and pyrrolidinyl groups;
the groups A and B possibly bearing 1 to 3 substituents chosen from a C₁-C₆ alkyl group, a C₁-C₆ alkoxy group, a C₆-C₁₄ aryl group, a heteroaryl group chosen from pyridyl, pyrimidyl, pyrrolyl, furyl and thienyl, a (C₆-C₁₄)aryl(C₁-C₆)alkyl group, a (C₆-C₁₄)aryl(C₁-C₆)alkyl(C₆-C₁₄)aryl group, a halogen or a trifluoromethyl, trifluoromethoxy, cyano, hydroxyl, nitro, amino, carboxyl, (C₁-C₆)alkoxycarbonyl, carbamoyl, (C₁-C₆)alkylsulfonyl, sulfoamino, (C₁-C₆)alkylsulfonylamino, sulfamoyl or (C₁-C₆)alkylcarbonylamino group;
or two of the substituents forming a methylenedioxy group, a solvate thereof or a salt of this acid.

2. Composition according to claim 1, characterised in that the glitazone is a compound of the general formula (11) below: 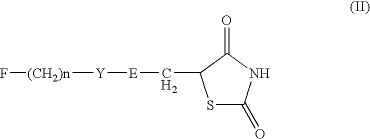
in which:
E represents a monocyclic, bicyclic or tricyclic aromatic hydrocarbon-based structure that can include one or more hetero atoms, this structure possibly being substituted by at least one (C₁-C₆) alkyl or acetyl radical, or possibly forming a 5- or 6-membered ring with the methylene radical attached to Y,
n is equal to 1, 2 or 3,
Y represents an oxygen atom, an —NHCO—, —CONH— or —CO— function; and
F features an amino group or an aromatic or non-aromatic, cyclic or bicyclic hydrocarbon-based group, optionally containing a hetero atom chosen from oxygen and nitrogen, the amino and hydrocarbon-based groups possibly containing at least one substitution chosen from a (C₁-C₆) alkyl radical, a halogen atom, an aryl or heteroaryl radical, an acetyl radical and a trifluoromethyl radical,
or a pharmaceutically acceptable salt thereof.

3. Composition according to claim 1, for treating diabetes.

4. Composition according to claim 3, for treating non-insulin-dependent diabetes.

5. Composition according to claim 1, for treating at least one of the pathologies associated with insulin resistance syndrome, more particularly chosen from dyslipidaemia, obesity, arterial hypertension, and microvascular and macrovascular complications, for instance atherosclerosis, retinopathies, nephropathies and neuropathies.

6. Pharmaceutical composition according to claim 1, characterised in that the weight ratio of the glitazone to the compound of the formula (I) ranges from 10⁻³ to 40, preferably from 10⁻³ to 10 and better still from 10⁻³ to 1.

7. Pharmaceutical composition according to claim 1, characterised in that the glitazone is chosen from rosiglitazone, pioglitazone, isaglitazone, KRP 297, CS 011, T 174, NP 0110, englitazone, darglitazone and ciglitazone.

8. Pharmaceutical composition according to claim 1, characterised in that the glitazone is chosen from rosiglitazone, pioglitazone, isaglitazone and KRP 297.

9. Composition according to claim 1, characterised in that the compound of the formula (I) is chosen from:
2-benzyl-4-(4-methoxyphenyl)-4-oxobutanoic acid
2-benzyl-4-(4-fluorophenyl)-4-oxobutanoic acid
2-cyclohexylmethyl-4-(4-methoxyphenyl)-4-oxobutanoic acid
2-benzyl-4-phenyl-4-oxobutanoic acid
2-(β-naphthylmethyl)-4-phenyl-4-oxobutanoic acid
2-benzyl-4-(β-naphthyl)-4-oxobutanoic acid
2-[(4-chlorophenyl)methyl]-4-(4-methoxyphenyl)-4-oxobutanoic acid
2-benzyl-4-(4-methylphenyl)-4-oxobutanoic acid
4-(4-fluorophenyl)-2-[(4-methoxyphenyl)methyl]-4-oxobutanoic acid
2-benzyl-4-(3,4-methylenedioxyphenyl)-4-oxobutanoic acid
2-benzyl-4-cyclohexyl-4-oxobutanoic acid
4-phenyl-2-[(tetrahydrofur-2-yl)methyl]-4-oxobutanoic acid,
the solvates, enantiomers and salts of these acids.

10. Composition according to claim 9, characterised in that the compound of the formula (I) is chosen from:
(−)-2-benzyl-4-(4-methoxyphenyl)-4-oxobutanoic acid
(+)-2-benzyl-4-(4-methoxyphenyl)-4-oxobutanoic acid
(−)-2-benzyl-4-(4-fluorophenyl)-4-oxobutanoic acid
(+)-2-benzyl-4-(4-fluorophenyl)-4-oxobutanoic acid.

11. Composition according to claim 1, which is suitable for oral administration.

12. Use of a glitazone in combination with a compound of the formula (I) as defined in claim 1 for the preparation of a medicinal combination for treating diabetes.

13. Use according to claim 12 for the preparation of a medicinal combination for treating non-insulin-dependent diabetes.

14. Use of a glitazone in combination with a compound of the formula (I) as defined in claim 1 for the preparation of a medicinal combination for treating at least one of the pathologies associated with insulin resistance syndrome, more particularly chosen from dyslipidaemia, obesity, arterial hypertension, and microvascular and macrovascular complications, for instance atherosclerosis, retinopathies, nephropathies and neuropathies.

15. Use according to claim 12, characterised in that the glitazone is of the formula (II).

16. Use according to claim 12, characterised in that the glitazone is chosen from rosiglitazone, pioglitazone, isaglitazone, KRP 297, CS 011, T 174, NP 0110, englitazone, darglitazone and ciglitazone.

17. Use according to claim 12, characterised in that the compound of the formula (I) is chosen from:
2-benzyl-4-(4-methoxyphenyl)-4-oxobutanoic acid
2-benzyl-4-(4-fluorophenyl)-4-oxobutanoic acid
2-cyclohexylmethyl-4-(4-methoxyphenyl)-4-oxobutanoic acid
2-benzyl-4-phenyl-4-oxobutanoic acid
2-(β-naphthylmethyl)-4-phenyl-4-oxobutanoic acid
2-benzyl-4-(β-naphthyl)-4-oxobutanoic acid
2-[(4-chlorophenyl)methyl]-4-(4-methoxyphenyl)-4-oxobutanoic acid
2-benzyl-4-(4-methylphenyl)-4-oxobutanoic acid
4-(4-fluorophenyl)-2-[(4-methoxyphenyl)methyl]-4-oxobutanoic acid
2-benzyl-4-(3,4-methylenedioxyphenyl)-4-oxobutanoic acid
2-benzyl-4-cyclohexyl-4-oxobutanoic acid
4-phenyl-2-[(tetrahydrofur-2-yl)methyl]-4-oxobutanoic acid,
the solvates, enantiomers and salts of these acids.

18. Use according to claim 12, characterised in that the medicinal combination is in the form of a unit dose comprising a glitazone and a compound of the formula (I) as defined in claim 1.

19. Use according to claim 18, characterised in that the unit dose comprises from 1 mg to 1 g of glitazone and from 12.5 to 400 mg of a compound of the formula (I).
